(19) United States
(12) Patent Application Publication
(10) Pub. No.: US 2026/0000328 A1
(43) Pub. Date: Jan. 1, 2026

(54) ARTIFICIAL INTELLIGENCE SYSTEMS FOR AUTOMATED COGNITIVE STATE ANALYSIS

(71) Applicant: UNIVERSIDAD DE VIGO, Vigo (ES)

(72) Inventors: Francisco Javier González Castaño, Vigo (ES); Francisco de Arriba Pérez, Vigo (ES); Silvia Garcia Méndez, Randufe-Tui (ES)

(73) Assignee: UNIVERSIDAD DE VIGO, Vigo (ES)

(21) Appl. No.: 19/243,415

(22) Filed: Jun. 19, 2025

Related U.S. Application Data
(60) Provisional application No. 63/664,578, filed on Jun. 26, 2024.

(51) Int. Cl. A61B 5/16; A61B 5/00; G06F 40/35

(52) U.S. Cl. CPC A61B 5/165; A61B 5/4803; A61B 5/7267; G06F 40/35

(57) ABSTRACT

Systems for automated cognitive state analysis including a computer terminal, a conversation assistant, a dialogue module, and a detection module. The computer terminal executes programmed instructions, receives user messages, and communicates system messages. The conversation assistant is in data communication with the computer terminal and generates system messages communicated by the computer terminal. The conversation assistant automatically generates system messages based on user messages received by the computer terminal and communicated to the conversation assistant. The dialogue module is in data communication with the computer terminal and analyzes the user-system dialogue to generate dialogue metrics based on the analysis of the user-system dialogue. The detection module is in data communication with the dialogue module and evaluates the dialogue metrics according to a cognitive state standard test and produces a cognitive state assessment based on the evaluation of the dialogue metrics to the cognitive state standard test.

### CROSS REFERENCE TO RELATED APPLICATIONS

This application claims priority to copending U.S. Application Ser. No. 63/664,578, filed on Jun. 26, 2024, which is hereby incorporated by reference for all purposes.

### BACKGROUND

The present disclosure relates generally to artificial intelligence systems generating natural language dialogues. In particular, artificial intelligence systems for automatically analyzing a person's cognitive state via natural language dialogue between the person and a conversation assistant are described.

Recent advances in artificial intelligence have enabled conversation assistants, also known as chatbots, to automatically generate responses to user requests. Conversation assistants are currently used in business, education, and medicine. However, imitating human intelligence is still beyond their reach. Consequently, conversation assistants are used for light entertainment or highly specialized tasks.

Users find it easier to communicate with a conversation assistant if the conversation assistant employs brief utterances. Conversation assistants employing brief utterances have been shown to keep users engaged for longer. Thus, conversation assistant interactions in use currently are usually precise and brief.

In the health field, there are online platforms for general medical care through which doctors and patients interact. Doctor and patient interactions on online platforms typically utilize one or more manual methodologies. Common manual methodologies include the Mini-Mental, Yesavage, and Goldberg cognitive, anxiety, and depression tests.

Conducting cognitive, anxiety, and depression tests under medical supervision suffers from white coat syndrome. White coat syndrome is a patient responding differently when in the presence of medical personnel compared to how the person would respond in a more comfortable, home setting.

An artificial assistant can replace a human medical professional when conducting cognitive, anxiety, and depression tests. However, utilizing an artificial assistant in place of a person requires the patient's cooperation and, as the patient feels under observation, does not completely eliminate the white coat syndrome.

To fully prevent white coat syndrome, the cognitive, anxiety, and depression tests must be conducted without the patient's awareness. Conducting such tests without the patient's awareness can be achieved by analyzing the patient's speech, applying machine learning techniques to features derived from fundamental components of the voice or its transcription into written language. However, this type of detection is limited by the intrinsic possibilities of the user's vocal expression and does not consider how the user reacts to a context or follows the thread of a dialogue.

Existing digital systems for automatic cognitive assessment derive features solely from the users' expressions and do not take into account the context of the conversations in which these expressions occur. For example, conventional approaches do not account for a user's coherence with the context or the user's ability to mention or recall things that were previously said. These limitations reduce the effectiveness of conventional digital systems for cognitive assessment compared to an expert human evaluator.

Large language models are close to enabling artificial conversation assistants to emulate a human in informal chats. However, even though conversation assistants can be trained to present step-by-step cognitive tests (as just another digital interface for these tests), they are still not capable of inferring a person's cognitive state by analyzing a dialogue session as a whole, the way another person would do it.

It would be desirable to have artificial intelligence systems that addressed the limitations of the prior art described above. In particular, it would be beneficial to have an artificial intelligence system capable of analyzing a user's mental state more consistently and objectively than is currently possible. It would be advantageous if an artificial intelligence system executed methods enabling analysis of a user's mental state based on interactions with a conversation assistant in a manner that was unapparent to the user to reduce or eliminate white coat syndrome.

Thus, there exists a need for artificial intelligence systems for automatically analyzing the cognitive state of a user that improve upon and advance the design of known approaches for analyzing a person's cognitive state. Examples of new and useful artificial intelligence systems relevant to the needs existing in the field of cognitive state analysis are discussed below.

Documents relevant to the background information above and to analyzing the cognitive state of a person in general are listed in the Relevant Documents section below. The complete disclosures of the documents listed in the Relevant Documents section are incorporated herein by reference for all purposes.

### SUMMARY

The present disclosure is directed to systems for automated cognitive state analysis. The systems include a computer terminal, a conversation assistant, a dialogue module, and a detection module.

The computer terminal is configured to execute programmed instructions, receive user messages from a user via text input or spoken words, and communicate system messages as displayed text or audible utterances or both in response to programmed instructions. The system messages and the user messages collectively define a user-system dialogue.

The conversation assistant is in data communication with the computer terminal and is defined by programmed instructions operable to generate system messages communicated by the computer terminal as displayed text or audible utterances or both. The conversation assistant is configured to automatically generate system messages based on user messages received by the computer terminal and communicated to the conversation assistant.

The dialogue module is in data communication with the computer terminal. The dialogue module is defined by programmed instructions operable to analyze the user-system dialogue to generate dialogue metrics based on the analysis of the user-system dialogue.

The detection module is in data communication with the dialogue module. The detection module is defined by programmed instructions operable to evaluate the dialogue metrics according to a cognitive state standard test and to produce a cognitive state assessment based on the comparison of the dialogue metrics to the cognitive state standard test.

### BRIEF DESCRIPTION OF THE DRAWINGS

FIG. 1 is a view of a conventional, human-administered analysis of a person's cognitive state, which is subject to skewed results due to white coat syndrome.

FIG. 2 is a view of a person utilizing a system for automated cognitive state analysis that avoids white coat syndrome.

FIG. 3 is a schematic view of another example of a system for automated cognitive state analysis engaging in a dialogue with a user via a terminal of the system.

FIG. 4 is a schematic view of a semantic module of the system shown in FIG. 3 extracting conversation context information from the dialogue between the user and the system.

FIG. 5 is a schematic view of a dialogue module and a detection module of the system shown in FIG. 3 cooperating to evaluate conversation context information.

FIG. 6 is a schematic view of an evocation module of the system shown in FIG. 3 evaluating the user's memory of selected key words from the dialogue between the user and the system.

### DETAILED DESCRIPTION

The disclosed systems for automated cognitive state analysis will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description.

Throughout the following detailed description, examples of various systems for automated cognitive state analysis are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire-based connector, whether directly or indirectly through a communication network.

“Controllably coupled” means that an electronic device controls operation of another electronic device.

### REFERENCE NUMBERS GLOSSARY

100: A system for automated cognitive state analysis.
101: A computer terminal used by a user to interact with the system.
102: A person using the system.
103: Messages produced by the conversation assistant, which may be utterances or displayed text and are designated as system messages.
104: Messages produced by the user, which may be utterances or displayed text and are designated as user messages.
105: User-system dialogue resulting from an exchange of system messages and user messages produced at different moments n, such as moments n, n−1, and n-m.
106: A conversation assistant.
107: A dialogue module.
108: A detection module for detecting condition C at moment n based on condition C model MCn−1 supplied by a model updater module.
109: A collection of system components.
202: An artificial intelligence model or module that checks for a semantic relationship A between user messages and system messages at different moments in view of the user-system dialogue.
203: A statistics generator that updates the statistics of semantic relationship A for the user given past history of semantic relationship A values.
204: Database of historical semantic relationship A compliance data for the user.
206: A metrics generator for semantic relationship A.
206b: Metrics of semantic relationship A at moment n for the user, designated as FAUn, generated by the metrics generator.
303: A metrics generator for semantic relationship B.
303b: Metrics of semantic relationship B for the user at instant n, designated as FBUn, generated by the metrics generator.
304: A linguistic metrics generator.
304b: Linguistic metrics for the user at instant n, designated as FLUn, generated by the linguistic metrics generator.
305: A word embedding generator.
305b: Word embeddings for the user at instant n, designated as WEUn, generated by the word embedding generator.
306: A metadata module.
306b: User metadata.
307: A condition tag generator.
307b: A condition tag with data about the user at instant n, designated as TUn, generated by the condition tag generator.
308: A model updater module.
308b: A model to detect condition C at instant n, designated as MCn, supplied by the model updater module.
308c: A model to detect condition C at instant n−1, designated as MCn−1, supplied by the model updater module.
310: A mental condition value detected by the detection module at instant n indicating the likelihood of the user having condition C, designated as CUn, based on processing inputs FAUn, FBUn, FLUn, and WEUn from the dialogue module using model MCn.
402: A system message communicating a recall statement to the user including selected words.
403: A system message communicating a test question to the user referencing the selected words included in the recall statement.
404: A user message responding to the test question posed in a prior system message.
406: An evocation module to check semantic evocation in the user-system dialogue.
407: A word extractor module that extracts selected words from the user-system dialogue for the recall statement and the test question.
408: A evocation detector module that analyzes semantic evocation of the user-system dialogue in view of the selected words extracted by the word extractor module.
409: An evocation score generated by the evocation module based on the analysis of the evocation detector module.
500: A second example of a system for automated cognitive state analysis.
501: A terminal in the form of a smartphone.
509: A collection of system components located on a remote server.

### Systems for Automated Cognitive State Analysis

With reference to the figures, systems for automated cognitive state analysis will now be described. The systems discussed herein function to analyze users' cognitive capacity and other mental conditions without requiring a professional's assistance. In some applications, the novel artificial intelligence systems are utilized by users from their homes.

The systems described herein are suitable for users who are not yet diagnosed with cognitive decline or mental issues, but who are concerned about their health. Additionally or alternatively, the novel systems are a resource for family and friends who might be concerned about a person's cognitive state, such as users in the phase of subjective cognitive decline. Importantly, the novel systems provide important information about a person's mental state without overburdening the healthcare system.

The novel systems numerically evaluate a user's cognitive capacity and other mental conditions based on dialogue between the user and a conversation assistant. The systems are configured to establish dialogue about topics of interest to the user. For example, the novel systems may engage in dialogues with a user about news topics, informational topics, entertainment topics, or no specific topic at all.

The reader will appreciate from the figures and description below that the presently disclosed systems address many of the shortcomings of conventional approaches to analyzing a user's cognitive state. For example, the novel systems described herein are capable of analyzing a user's mental state more consistently and objectively than is currently possible. Using the novel systems disclosed herein that utilize artificial intelligence and automated processes reduces or completely avoids human bias and human error in the cognitive state analysis process.

Advantageously, the novel systems described below enable analysis of a user's mental state based on interactions with a conversation assistant in a manner that is unapparent to the user. For example, the novel systems may accomplish its automated analysis as the system and a user discuss topics of interest to the user, engage in entertainment activities (such as a voice-controlled game), or discuss news topics.

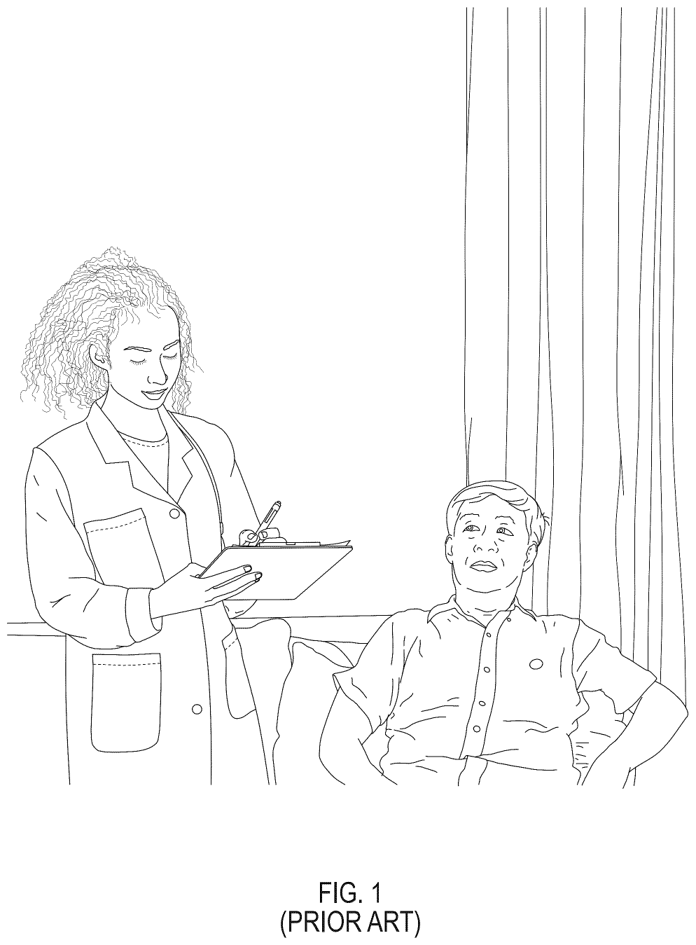

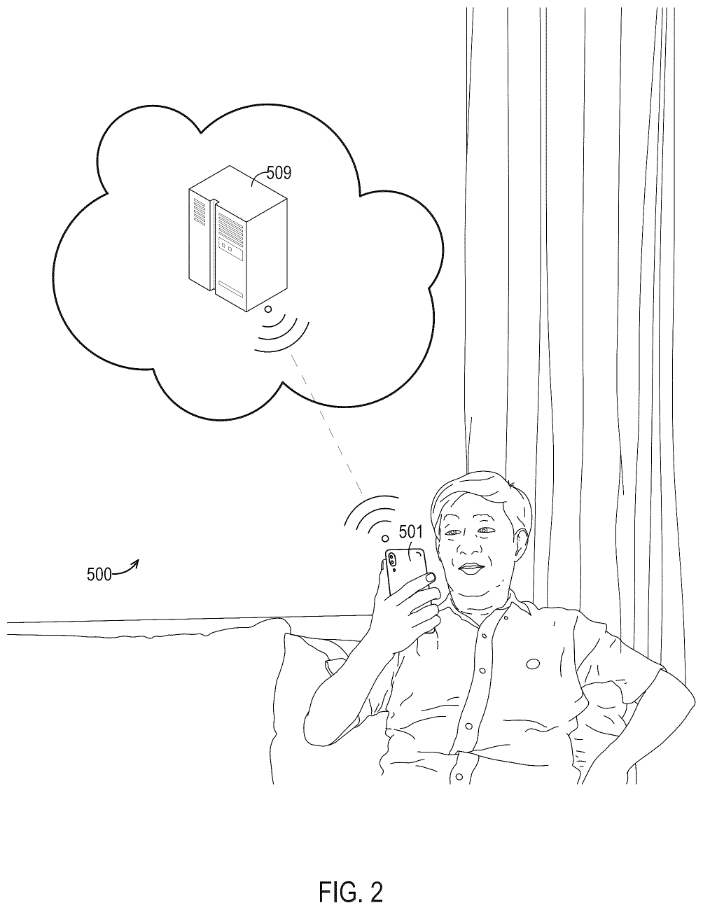

Significantly, and in contrast to human administered cognitive assessments like shown in FIG. 1, cognitive state evaluation with the novel systems herein may occur without the user being aware that he or she is being evaluated, such as depicted in FIG. 2. As shown in FIG. 2, the cognitive state evaluation can occur in a friendly environment, such as the person's home, with a familiar device like a smartphone 501 controllably coupled to other components 509 of an automated system 500 operating on a remote server computer. As a result of the analysis taking place in a low stress environment and in an unapparent way, the novel systems reduce or eliminate white coat syndrome, which is a prevalent issue with conventional approaches to analyzing a user's cognitive state.

Beneficially, the novel systems engage in dialogue with the user via artificial intelligence communication models. The novel systems' dialogue capabilities improve over conventional cognitive state analysis solutions by enabling the novel systems to consider context relevant to the dialogue. In contrast to conventional solutions that rely on just users' expressions, the novel systems extract information from the users' expressions and their relationship with the context and the flow of the conversation.

The novel systems are configured to analyze the contextual information extracted from conversations with a user. In particular, the novel systems input the contextual information extracted from conversations into artificial intelligence models to estimate the user's cognitive state and other related mental conditions, such as levels of anxiety and depression.

### System Embodiment One

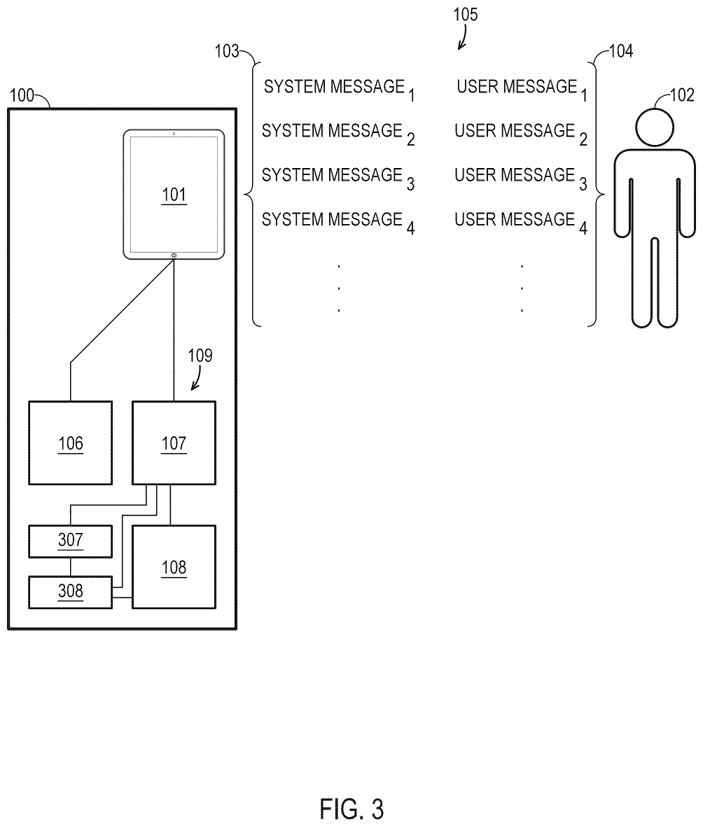

With reference to FIGS. 3-6, a first example of a system for automated cognitive state analysis, system 100, will now be described. As shown in FIG. 3, system 100 includes a computer terminal 101, a conversation assistant 106, a dialogue module 107, a detection module 108, a tagger module 307, and a condition model updater module 308. Conversation assistant 106, dialogue module 107, detection module 108, tagger module 307, and condition model updater module 308 define a collection 109 of components of system 100. The components of system 100 are described further below.

In some examples, the systems for automated cognitive state analysis include fewer components than shown in FIGS. 3-6, such as not including a tagger module or a condition model updater module. In other examples, the systems include additional or alternative features.

System 100 analyzes dialogue between conversation assistant 106 and a user 102. The analysis performed on the dialogue by system 100 serves to check if the user's message has certain semantic relationships with previous dialogue, such as coherence, consistency, information retrieval, agreement, disagreement, or other relationships.

Messages of user 102 are analyzed by system 100 together with prior messages produced by conversation assistant 106. Messages by conversation assistant 106 immediately preceding responses of user 102 may be most relevant to the significance of a user's message at a given moment. Additionally or alternatively, system 100 may compare a user's response to earlier dialogue content from either user 102 or conversation assistant 106.

The analyses of semantic relationships can produce numerical values reflecting compliance with the semantic relationships. For example, a zero (0) value may indicate non-compliance and a value of one (1) may indicate compliance. Instead of binary numbers, the numerical values may be a numerical scale that reflects the degree of compliance.

The semantic relationship compliance values facilitate training a detector module 108. Detector module 108 detects cognitive states and other mental conditions of user 102 by comparing the compliance values to a sample of people representative of those states and conditions. Cognitive state examples include cognitive impairment versus non-impairment, or depression versus non-depression. Training detector module 108 may also utilize values obtained exclusively from messages of user 102, such as fundamental components of voice, emotion, sentiment, types of constituent words, word embeddings, etc.

System 100 is configured to employ artificial intelligence techniques based on class separation in a space. With class separation techniques, a measure of the separation of a user's values from his or her class provides a degree of compliance with the estimated class on a continuous scale.

The separation measure may be the distance from a user's representative vector to a separating surface. A high value of the separation measure may correspond to a high degree of compliance. Additionally, by retraining the model multiple times with different datasets, the reliability of the estimation for a particular user can be assessed from the statistical distribution of class assignments.

The systems described herein make it possible to reduce care resources allocated to mental patients. System 100 is conveniently implemented on a handheld electronic device of the user, terminal 101. Additionally or alternatively, like shown in FIG. 2, a collection of system components may be located and executed on a remote server computer in data communication with a user's personal device. The automated evaluations performed by the systems described herein are based on dialogues of interest to the users with a conversation assistant capable of running on the users' personal terminals.

### Computer Terminal

As shown in FIG. 3, computer terminal (hereinafter simply terminal) 101 enables user 102 to interact with conversation assistant 106 along with other components of system 100, such as dialogue module 107 and detection module 108. Similarly, FIG. 2 depicts a user interacting with terminal 501 to access components 509 of system 500 for automated cognitive state analysis. With reference again to FIG. 3, terminal 101 presents user 102 with a system interface to facilitate interactions between system 100 and user 102.

In the present example, user 102 uses terminal 101 to execute software embodying conversation assistant 106 stored in memory on terminal 101. In other examples, such as shown in FIG. 2, the user may use the terminal to exchange data with another computer hosting and executing the conversation assistant software. In automated system 500 shown in FIG. 2, a terminal 501 wirelessly exchanges data over a distributed data network with a remote computing device hosting system components 509, including a conversation assistant, a dialogue module, and a detection module.

Terminal 101 includes text input capabilities, a display, a microphone, and a speaker. The speaker enables terminal 101 to produce sounds, including sounds corresponding to system messages 103. The display enables terminal 101 to display system messages 103 in the form of text messages, images, or videos. The microphone enables terminal 101 to capture user messages 104 spoken by user 102. The text input capabilities of terminal 101, such as a physical keyboard, a touchscreen keyboard, and/or voice transcription software, enables terminal 101 to capture user messages 104 in the form of text input.

The terminal may be any currently known or later developed type of computing device suitable for running artificial intelligence programs and/or exchanging data with computing systems executing artificial intelligence programs. A wide range of computing devices are suitable for the terminal, including handheld computing devices, tablet computers, laptop computers, desktop computers, wearable computing devices, augmented reality computing devices, and virtual reality computing devices.

### Conversation Assistant

As shown in FIG. 3, conversation assistant 106 serves to engage in dialogue with user 102 via terminal 101. Conversation assistant 106 is defined by programmed instructions operable to generate system messages 103 communicated by terminal 101 as displayed text or audible utterances or both. Conversation assistant 106 is a software program that interacts with user 102 via voice utterances and/or textual displays to provide information to user 102 and to collect information from user 102. Voice utterances and textual displays are collectively referred to as messages in this document.

FIG. 3 shows conversation assistant 106 producing a series of system messages 103 at different moments 1, 2, 3, and 4, which are designated as system message, system message₂, etc. FIG. 3 further shows user 102 producing a series of user messages 104 at different moments 1, 2, 3, and 4, which are designated as user message₁, user message₂, etc. Back and forth exchanges of system messages 103 and user messages 104 between conversation assistant 106 and user 102 are referred to as user-system dialogue 105.

The information exchanged between conversation assistant 106 and user 102 in user-system dialogue 105 may include news, music, weather data, or any other topic or data of interest. Some well-known examples of conventional conversation assistants are Google Assistant and Alexa. The conversation assistant may be any currently known or later developed software program facilitating automated communication.

User-system dialogue 105 enables system 100 to analyze the cognitive state of user 102. System 100 analyzes user-system dialogue 105 in a variety of ways to assess the cognitive state of user 102.

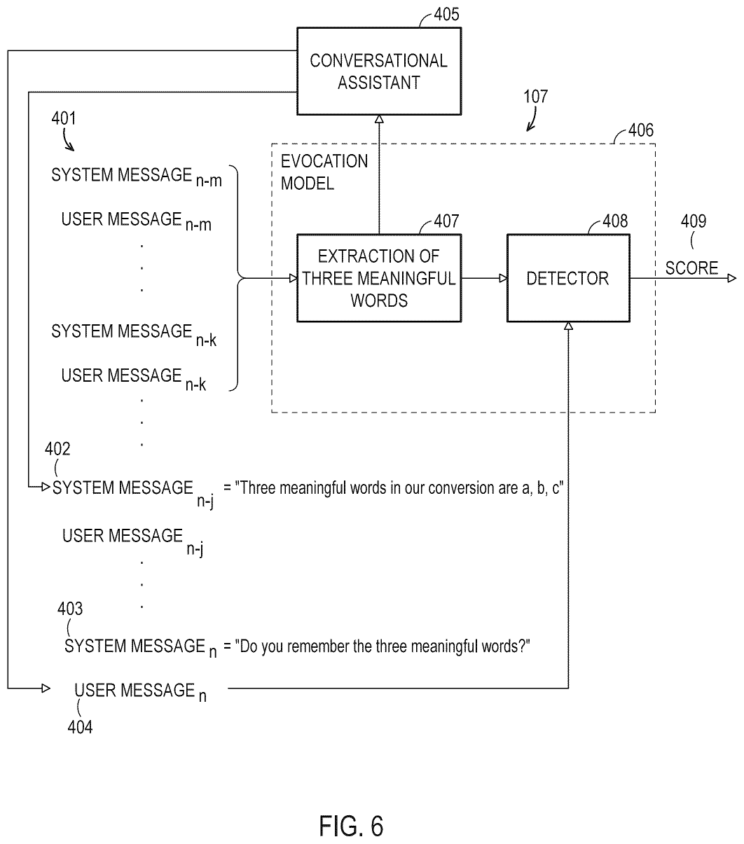

One way system 100 utilizes user-system dialogue 105 to assess the cognitive state of user 102 is shown in FIG. 6. With reference to FIG. 6, system 100 considers answers provided by user 102 to questions posed by system 100 within user-system dialogue 105. In the example shown in FIGS. 3-6, conversation assistant 106 is configured to intersperse simple questions, such as question 403, directed to user 102 within user-system dialogue 105 based on the information provided by user 102. The questions presented to user 102 by conversation assistant 106 may be based on the Mini-Mental State Examination, the Goldberg scale, the Yesavage test, or others.

Questions, such as question 403, presented by conversation assistant 106 are generated automatically from phrases in user messages 104. For example, with continued reference to FIG. 6, a word extractor module 407 of an evocation module 406 is configured to identify keywords in user messages 104, such as places or proper names, and to automatically generate questions 403 directed to those keywords. Additionally or alternatively, the evocation module may be configured to recognize and generate questions based on other words deemed related to the keywords extracted by the word extractor module through automatic co-reference analysis.

As described above, system messages 103 may be voice utterances or textual displays. Utterances can be synthetic voice produced through a speaker or headphones of terminal 101. The text can be displayed on a screen of terminal 101, either separately or simultaneously with utterances delivered audibly.

In response to system messages 103, user 102 responds successively with his or her user messages 104, which may be utterances or text inputs. User messages 104 in the form of utterances can be the voice of user 102 captured by a microphone of terminal 101. User messages 104 in the form of text can be text entered by user 102 into terminal 101. Additionally or alternatively, user messages 104 in the form of text may derive from automatically transcribing words spoken by user 102 to text.

### Dialogue Module

Dialogue module 107 functions to generate data from user-system dialogue 105 for detection module 108 to analyze when assessing to what extent user 104 has a given mental condition. Dialogue module 107 is defined by programmed instructions. The programmed instructions are operable to analyze user-system dialogue 105 and to generate data in the form of dialogue metrics based on the analysis of user-system dialogue 105.

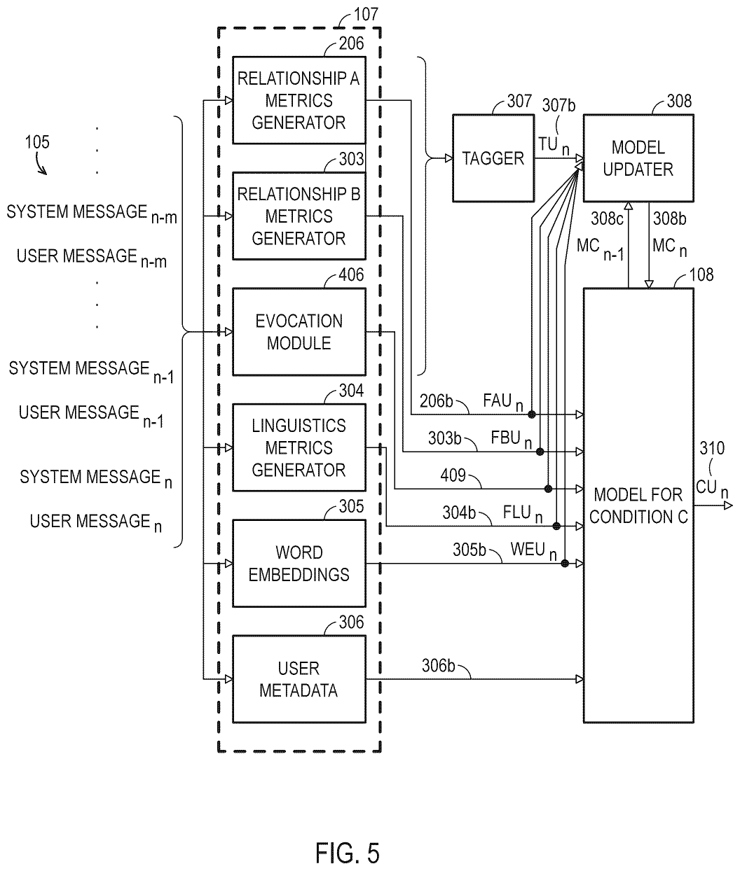

The dialogue metrics data generated by dialogue module 107 are further utilized by tagger module 307 to add mental condition tags to the data. Model updater 308 also uses the dialogue metrics data to update how detection module 108 evaluates the extent to which a mental condition is present. FIG. 5 schematically shows dialogue module 107 outputting dialogue metrics data to tagger module 307, model updater 308, and detection module 108.

In the present example, as shown in FIG. 5, dialogue module 107 includes a first semantic module 206, a second semantic module 303 a third semantic module 406, a linguistics module 304, and a metadata module 306. First, second, and third semantic modules 206, 303, and 406 are also referred to as first, second, and third semantic metrics generators. Linguistics module 304 is also referred to as a linguistic metrics generator.

In other examples, the dialogue module includes a subset of the components of dialogue module 107. In certain examples, the dialogue module includes additional or alternative components. The components of dialogue module 107 and the different dialogue metrics generated by the components are discussed in the sections below.

### Semantic Metrics Generators

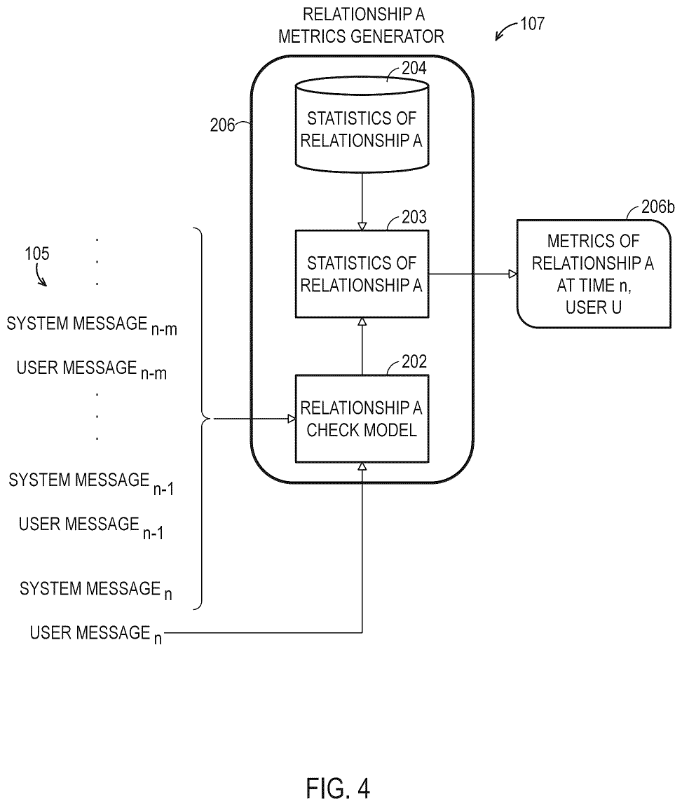

First, second, and third semantic metrics generators 206, 303, and 406 generate semantic metrics based on different semantic relationships, which are defined by semantic relationship rules. For example, FIG. 4 depicts a semantic metrics generator 206 generating semantic metrics 206b based on a semantic relationship rule A at instant n for user 102. Second semantic relationship generator 303 generates semantic metrics 303b based on a semantic relationship rule B. FIGS. 5 and 6 depicts third semantic metrics generator 406 (discussed in more detail in a separate section below) generating semantic metrics 409 based on a semantic relationship rule of evocation.

Semantic module 206 generates semantic metrics 206b by analyzing user-system dialogue 105. Semantic module 206 generates semantic metrics 206b dynamically in real time as user-system dialogue 105 progresses. System messages 103 and user messages 104 making up user-system dialogue 105 occur at moment n and prior. The semantic relationship analyzed by semantic metrics generator 206 exhibited in user-system dialogue 105 can include concordance (gender, number, temporal, etc.), coherence, agreement, opposition, order inversion, repetition, evocation, or any other relationship.

With reference to FIGS. 4 and 5, semantic metrics generator 206 generates numerical metrics 206b related to compliance with a semantic relationship A between different messages within user-system dialogue 105. In FIG. 5, the label FAUn with reference number 206b represents the set of semantic metrics for user 102 at moment n linked to semantic relationship A. Further in FIG. 5, the label FBUn with reference number 303b represents a set of semantic metrics for user 102 at moment n linked to semantic relationship B. Semantic metrics generator 206 is specific to semantic relationship A where A identifies a particular semantic relationship.

In the example shown in FIG. 4, semantic metrics generator 206 includes an artificial intelligence module 202, a statistics generator 203, and a compliance database 204. In other examples, the semantic metrics generator includes a subset of the components of semantic metrics generator 206. In certain examples, the semantic metrics generator includes additional or alternative components. The components of semantic metrics generator 206 are described in the sections below.

### Artificial Intelligence Module

Artificial intelligence module 202 checks whether a given section of user-system dialogue 105 complies with semantic relationship A. As depicted in FIG. 4, user-system dialogue 105 includes user message n, which is the user's latest message received by system 100. As further shown in FIG. 4, user-system dialogue 105 includes user messages n−1 and n-m, which are the user's prior messages received by system 100. User-system dialogue 105 also includes system messages n, n−1, and n-m, which are all or part of the previous m system messages of conversation assistant 106.

The artificial intelligence module can be a large language model, a neural network, a support vector machine, an ensemble of decision trees, or any other artificial intelligence technique. In different embodiments, the degree of compliance can be expressed as a binary variable, a discrete variable, or a continuous variable. A continuous variable may represent the distance to a separation surface in a feature space or as an estimation of the probability of compliance with the semantic relationship.

### Statistics Generator

Statistics generator 203 is configured to generate statistics related to the degree of compliance between user-system dialogue 105 and semantic relationship A at moment n measured by artificial intelligence module 202. Statistics generator 203 is also configured to consider previous moments when generating statistics related to compliance between user-system dialogue 105 and semantic relationship A.

The statistics generated by statistics generator 203 may include a wide variety of statistical values. For example, the statistical values may include the last k values of the degree of compliance, or their maximum, minimum, mean, or median values. In some examples, the statistical values include representative values of different quartiles or deciles. The statistical values generated by statistics generator 203 may include any parameters defining any estimation of the probability distribution of the degree of compliance.

### Compliance Database

Compliance database 204 is configured to store the measures of compliance output by artificial intelligence module 202. Compliance database 204 is also configured to store statistical values generated by statistics generator 203.

The compliance database may be any currently known or later developed type of database, including relational records databases. The compliance database may be stored and executed local to other components of the system of may be stored and executed remotely and accessed over a distributed data network.

### Third Semantic Generator Evaluating Evocation

With reference to FIG. 6, third semantic metrics module 406 evaluating evocation will be described in more detail. A third semantic metrics module is optionally included in certain examples of the present systems for automated cognitive state analysis.

Third semantic metrics module 406 is defined by programmed instructions and assesses the semantic relationship of evocation. In the context of the presently described systems, evocation is a semantic relationship between a user message 404 in response to a prior system message 402 of conversation assistant 106. Evocation module 406 detects evocations from user-system dialogue 105 between user 102 and conversation assistant 106 at each instant n.

Thus, evocation module 406 functions as a semantic metrics generator for an evocation-oriented semantic relationship. In a sense, third semantic metrics module 406 fulfills a similar role as semantic relationship check module 202 included in semantic metrics generator 206.

As shown in FIG. 6, third semantic metrics module 406 includes a word extractor module 407 and an evocation detector module 408. Word extractor module 407 and evocation detector module 408 are in data communication with conversation assistant 106. Third semantic metrics module 406 processes user-system dialogue 105 received from conversation assistant 106 and supplies conversation assistant 106 with information to include in system messages 103, including system messages 402 and 403 shown in FIG. 6.

In the example shown in FIG. 6, word extractor module 407 extracts three meaningful words from user-system dialogue 105 between user 102 and conversation assistant 106. The number of meaningful words extracted could be larger or smaller than three words in other examples. The meaningful words may be established by inputting user-system dialogue 105 into a large language model along with an instruction prompt.

In response to instructions from third semantic metrics module 406, conversation assistant 106 informs user 102 about the extracted meaningful words in a system message 402, which defines a recall statement, during the course of the dialogue with user 102, such as at moment n-j. Later, conversation assistant 106 asks user 102 to repeat the meaningful words in a system message 403, which defines a test question.

Evocation detector module 408 receives and processes user message 404, which is the response of user 102 to test question 403 posed by conversation assistant 106. Evocation detector module 408 generates an evocation score 409 based on how well user message 404 demonstrates the user's ability to remember the meaningful words communicated in recall statement 402. The more words the user remembers in user message 404, the higher evocation score 409 will be.

In some examples, evocation score 409 is not utilized by detection module 108 to evaluate a mental state of user 102. Additionally or alternatively to detection module 108 utilizing evocation score 409 for mental state evaluation, evocation score 409 may be used by tagger module 307 to generate condition tags 307b. Model updater 308 may use condition tags 307b derived in part from evocation score 409 to retrain model 308b used by detection module 108 for condition C. Utilizing model updater 308 to retrain the detection model may be selectively performed when considered necessary or on a regular basis.

### Linguistics Module

Linguistics module 304 generates linguistic metrics 304b, which are used by detection module 108 to assess the extent to which user 102 has a given mental condition. Linguistics metrics 304b are also used by model updater 308 to update detection model 308b.

Linguistics module 304 is defined by programmed instructions. The programmed instructions of linguistics module 304 are operable to generate linguistics metrics 304b by comparing user-system dialogue 105 to a linguistic standard.

In FIG. 5, linguistics metrics 304b are designated with variable FLUn. Linguistic metrics 304b include the number of words of a certain type included in user messages 104. The linguistic metrics may include any other relevant linguistic metric as well.

### Word Embedding Module

With reference to FIG. 5, word embedding module 305 generates word embeddings 305b.

As shown in FIG. 5, word embeddings 305b are used by detection module 108 when evaluating whether user 102 exhibits a given cognitive state. As further shown in FIG. 5, word embeddings 305b are used to by model updater 308 to update models 308b supplied to detection module 108.

Word embeddings 305b facilitate encoding words, word components, or groups of words as separate data values. In FIG. 5, word embeddings 305b generated by word embedding module 305 at a given moment n are designated with the variable WEUn.

### Metadata Module

Metadata module 306 functions to supply detector module 108 with metadata 306b about user 102 to assist with detecting the cognitive condition of user 102. Metadata 306b supplied by metadata module 306 may be time-independent or time-dependent. In examples where the metadata is time-independent, detection module 108 may still consider metadata at each instant n.

### Detection Module

Detection module 108 (also referred to herein as a detection model) functions to detect the extent to which a mental condition is present or absent in user 102. Detection module 108 is defined by programmed instructions.

The programmed instructions of detection module 108 evaluate the dialogue metrics generated by dialogue module 107 according to a cognitive state standard test. The evaluation performed by detection module 108 produces a cognitive state assessment based on the comparison of the dialogue metrics to the cognitive state standard test.

FIG. 5 depicts detection module 108 detecting a mental condition C or its absence in user 102 at instant n. Mental condition C may correspond to cognitive impairment, anxiety, depression, or any other condition.

Data representing a cognitive state detection result is indicated with reference number 310 and nomenclature CUn. Detection module 108 is updated by model updater 308 at each instant n as indicated schematically with reference number 308b.

Detection module 108 receives various dialogue metric inputs from dialogue module 107 at each instant to facilitate detecting a mental condition C using detection model 308b supplied by model updater 308. As discussed above, dialogue module 107 includes various sub-modules that are each configured to output different types of data relevant for operating and updating detection module 108.

For example, detection module 108 receives semantic metrics 206b and 303b from first and second semantic modules 206 and 303, respectively, at each instant. Detection module 108 uses semantic metrics 206b and 303b as data inputs for detection model 308b to yield mental condition value 310 at each instant. Semantic metrics 206b and 303b result from the analysis of user-system dialogue 105 at moment n and prior by corresponding first and second semantic modules 206 and 303. The semantic relationships can include concordance (gender, number, temporal, etc.), coherence, agreement, opposition, order inversion, repetition, or any other relationship.

As shown in FIGS. 5 and 6, detection module 108 also receives and processes evocation score 409 from evocation module 406 at each instant. As described above, evocation score 409 is a particular type of semantic relationship. Detection module 108 applies evocation score 409 in detection model 308b to yield mental condition value 310.

In addition to metrics linked to semantic relationships, detection module 108 considers complementary data metrics generated by dialogue module 107 as well. For example, detection module 108 receives and considers linguistic metrics 304b, word embeddings 305b, and metadata 306b when evaluating mental condition value 310. Even though metadata 306b may be time-independent, detection module 108 may consider metadata at each instant n.

Based on all the data metrics output by dialogue module 107 at each instant n, detection module 108 evaluates the degree of compliance with mental condition C. Detection module 108 may be a large language model, a neural network, a support vector machine, an ensemble of decision trees, or any other artificial intelligence technique.

Detection module 108 expresses the degree of compliance with mental condition C for user U at moment n as a variable CUn, designated in FIG. 5 with reference number 310. Variable CUn can be represented as a binary variable, a discrete variable, or a continuous variable. Continuous variables may represent the distance to a separation surface in the data space. Variable CUn may further represent an estimation of the probability of compliance with the semantic relationship.

### Tagger Module

As shown in FIG. 5, tagger module 307 generates condition tags TUn 307b for user 102. In the present example, tagger module 307 updates condition tag 307b at each instant n. Condition tag 307b corresponds to condition C and is used by model updater 308 to update condition model 308b.

In more detail, once enough scores have been generated by dialogue module 107 for semantic relationships corresponding to a standard test for condition C at any instant n, tagger module 307 produces condition tag 307b for user 102 corresponding to condition C. For example, for the Mini-Mental State Examination (MMSE) for assessing cognitive impairment, some relevant semantic relationships include temporal concordance, location concordance, evocation, registration (equivalent to evocation for low j in FIG. 6, as registration consists in the ability to reproduce what has just been said), numerical correctness, repetition, and comprehension. Condition tag TUn is used to label all data metrics of user 102 until a new tag becomes available. Condition tag TUi+1 equals condition tag TUi until condition tag 307b is updated by tagger module 307.

### Condition Model Updater Module

As shown in FIG. 5, a model updater 308 is configured to produce a new condition model MCn 308b used by detection module 108 to detect the extent to which user 102 exhibits mental condition C. Producing new condition model 308b may be referred to as updating existing condition model 308c. Updating condition model 308c with new condition model 308b may occur at each instant n or may occur on demand when an operator determines that updating condition model 308c is worthwhile or necessary.

As shown in FIG. 5, model updater 308 generates an updated condition model 308b based on one or more data inputs. For example, model updater 308 may determine new condition model 308b based on metadata 306b; data metrics 206b, 303b, 304b, 305b, and/or 409; and/or condition tag 307b at instant n. Additionally or alternatively, model updater 308 may base updated condition model 308b on existing conditional model 308c for condition C, designated as MCn−1 in FIG. 5.

In some embodiments, model updater 308 generates new condition model 308b based entirely on new data generated by dialogue module at each instant n. In some examples, model updater 308 generates new condition model 308b based on a combination of new data generated at instant n and a subset of past data. Additionally or alternatively, module updater 308 may consider a corresponding user condition tag 307b with past or present data metrics when generating a new condition model.

### Methods of Automated Cognitive State Analysis

Various methods for automated cognitive state analysis are enabled by the systems described above. These methods are described below.

One method to assess the mental state of a user includes multiple steps, including generating system messages in the form of utterances or texts that are presented to a user with a conversation assistant via a terminal. In another step, the method includes capturing user messages in the form of utterances or texts produced by the user in response to the system messages of the conversation assistant.

A further step is applying an artificial intelligence model to measure a semantic relationship between the last user message and the previous user-system dialogue, that is, the previous exchange of system messages and user messages between the conversation assistant and the user. Another step in the method is keeping a database of past measurements of the semantic relationship.

The method continues with producing a set of data metrics, including the measurement of the semantic relationship and its statistics computed from the database of past measurements. A final step of the present method example is applying an artificial intelligence model to assess the user's mental state from the set of data metrics generated.

The method above may optionally include measuring another semantic relationship with at least one additional artificial intelligence model.

Alternatively, the method may include considering other sets of data taken from user metadata, linguistic properties, and word embeddings of the previous user-system dialogue.

In some examples, the artificial intelligence models for measuring semantic relationships instruct the conversation assistant to insert utterances or texts corresponding to standard mental state tests. In such examples, the mental state of a user may be labeled based on the output of the artificial intelligence models to measure semantic relationships, with these models receiving as input the user's utterances or texts in response to conversation assistant utterances or texts corresponding to standard tests of mental state.

Such methods may include retraining the artificial intelligence model used to assess the user's mental state from the set of dialogue metrics data. Retraining may be accomplished with labels based on the output of the artificial intelligence models used to measure semantic relationships when the labels become available. In such examples, the method may consider another set of characteristics taken from user metadata, linguistic properties, and word embeddings of the previous dialogue.

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

### RELEVANT DOCUMENTS

Xu, A., Liu, Z., Guo, Y., Sinha, V., & Akkiraju, R. (2017). A new chatbot for customer service on social media. In Proc. Conference on Human Factors in Computing Systems, pp. 3506-3510, 2017.
Thomas, N. T. (2016). An e-business chatbot using AIML and LSA. In Proc. International Conference on Advances in Computing, Communications and Informatics, pp. 2740-2742.
Robins, B., Dautenhahn, K., Te Boekhorst, R., & Billard, A. (2005). Robotic assistants in therapy and education of children with autism: can a small humanoid robot help encourage social interaction skills?. Universal access in the information society, 4 (2), 105-120.
Kumar, V., & Keerthana, A. (2016). Sanative Chatbot for Health Seekers. International Journal of Engineering and Computer Science, 5, pp. 16022-16025.
Hill, J., Randolph Ford, W., & Farreras, I. G. (2015). Real conversations with artificial intelligence: A comparison between human online conversations and human chatbot conversations. Computers in Human Behavior 49, 245-250.
Folstein, M., Folstein, S. E., & McHugh, P. R. (1975). Mini-Mental State, a Practical Method for Grading the Cognitive State of Patients for the Clinician. Journal of Psychiatric Research, 12 (3), 189-198.
Mario, B., Massimiliano, M., Chiara, M., Alessandro, S., Antonella, C., & Gianfranco, F. (2009). White-coat effect among older patients with suspected cognitive impairment: prevalence and clinical implications. International Journal of Geriatric Psychiatry: A Journal of the Psychiatry of Late Life and Allied Sciences, 24 (5), 509-517.
Method and server for dementia test based on questions and answers using artificial intelligence call. Patent EP4220648A1.
Cognitive function evaluation apparatus and cognitive function evaluation system. Patent JP2017213157A. Medical assessment based on voice. Patent US20230352194A1.
Systems and methods for digital speech-based evaluation of cognitive function, Patent WO2022212740A2.
García Méndez, S., de Arriba-Pérez, F., González Castaño, F. J., Regueiro Janeiro, J. A., & Gil-Castiñeira, F. (2021). Entertainment Chatbot for the Digital Inclusion of Elderly People without Abstraction Capabilities. IEEE Access 9, 75878-75891.
Yesavage, J. A., Brink, T. L., Rose, T. L., et al. (1982). Development and validation of a geriatric depression screening scale: a preliminary report. Journal of Psychiatric Research, 17 (1), 37-49.
Goldberg, D., Bridges, K., Duncan-Jones, P., & Grayson, D. (1988). Detecting anxiety and depression in general medical setting. BMJ (Clinical research ed.), 297, 897-9.

1. A system for automated cognitive state analysis, comprising:
a computer terminal configured to:
execute programmed instructions;
receive user messages from a user via text input or spoken words; and
communicate system messages as displayed text or audible utterances or both in response to programmed instructions, the system messages and the user messages collectively defining a user-system dialogue;
a conversation assistant in data communication with the computer terminal, the conversation assistant defined by programmed instructions operable to generate system messages communicated by the computer terminal as displayed text or audible utterances or both, the conversation assistant being configured to automatically generate system messages based on user messages received by the computer terminal and communicated to the conversation assistant;
a dialogue module in data communication with the computer terminal, the dialogue module defined by programmed instructions operable to analyze the user-system dialogue to generate dialogue metrics based on the analysis of the user-system dialogue; and
a detection module in data communication with the dialogue module, the detection module defined by programmed instructions operable to evaluate the dialogue metrics according to a cognitive state standard test and to produce a cognitive state assessment based on the evaluation of the dialogue metrics to the cognitive state standard test.

2. The system of claim 1, wherein the dialogue module includes a semantics module defined by programmed instructions operable to generate semantic metrics for use by the detection module by comparing the user-system dialogue to a semantic relationship rule.

3. The system of claim 2, wherein the semantics module is operable to generate semantic metrics dynamically in real time as the user-system dialogue progresses.

4. The system of claim 2, wherein:
the semantics module defines a first semantics module;
the semantics metrics defines first semantics metrics;
the semantic relationship rule defines a first semantic relationship rule; and
the dialogue module includes a second semantics module, the second semantics module defined by programmed instructions operable to generate second semantic metrics for use by the detection module to evaluate a second semantic relationship by comparing the user-system dialogue to a second semantic relationship rule.

5. The system of claim 2, wherein the semantics metrics are based at least in part on an automated assessment of concordance in the user-system dialogue.

6. The system of claim 2, wherein the semantics metrics are based at least in part on an automated assessment of coherence in the user-system dialogue.

7. The system of claim 2, wherein the semantics metrics are based at least in part on an automated assessment of agreement in the user-system dialogue.

8. The system of claim 2, wherein the semantics metrics are based at least in part on an automated assessment of opposition in the user-system dialogue.

9. The system of claim 2, wherein the semantics metrics are based at least in part on an automated assessment of order inversion in the user-system dialogue.

10. The system of claim 2, wherein the semantics metrics are based at least in part on an automated assessment of repetition in the user-system dialogue.

11. The system of claim 2, wherein the dialogue module includes a second semantic metrics module in data communication with the conversation assistant, the second semantic metrics module being defined by programmed instructions operable to:
instruct the conversation assistant to communicate a test question to the user via the computer terminal;
assess semantic evocation from the test question and a test answer communicated by the user in response to the test question; and
generate evocation metrics for use by the detection module based on the assessment of semantic evocation between the test question and the test answer.

12. The system of claim 11, wherein the programmed instructions of the second semantic metrics module are operable to extract selected words from the user-system dialogue for use when assessing sematic evocation.

13. The system of claim 12, wherein the programmed instructions of the second semantic metrics module are further operable to instruct the conversation assistant to communicate a recall statement referencing the selected words.

14. The system of claim 13, wherein the test question references the recall statement.

15. The system of claim 14, wherein the test question asks the user to recall the selected words referenced in the recall statement.

16. The system of claim 1, wherein the dialogue module includes a linguistics module defined by programmed instructions operable to generate linguistics metrics for use by the detection module by comparing the user-system dialogue to a linguistic standard.

17. The system of claim 1, wherein the dialogue module includes a metadata module defined by programmed instructions operable to obtain metadata about the user for use by the detection module.

18. The system of claim 1, further comprising a tagger module in data communication with the dialogue module and the detection module, the tagger module defined by programmed instructions operable to generate condition tags indicating the extent to which a user exhibits a given cognitive state condition.

19. The system of claim 1, wherein the programmed instructions defining the detection module include artificial intelligence instructions based on one or more of large language models, support vector machines, and decision tree ensembles.

20. The system of claim 1, further comprising a condition model updater module in data communication with the detection module, the condition model updater module being defined by programmed instructions operable to dynamically modify how the detection module produces cognitive state assessments based on prior dialogue metrics generated by the dialogue module and previous condition tags generated by a tagger module.
